(19) United States
(12) Patent Application Publication
(10) Pub. No.: US 2024/0093039 A1
(43) Pub. Date: Mar. 21, 2024

(54) WOOD-PRESERVING TREATMENT SOLUTION AND METHOD FOR PRODUCING WOOD PROVIDED WITH WOOD PRESERVATIVE

(71) Applicant: AGC INC., Tokyo (JP)

(72) Inventors: Hiroaki MITSUOKA, Chiyoda-ku (JP); Hiroki HAYAMIZU, Chiyoda-ku (JP)

(73) Assignee: AGC INC., Tokyo (JP)

(21) Appl. No.: 18/510,739

(22) Filed: Nov. 16, 2023

Related U.S. Application Data
(63) Continuation of application No. PCT/JP2022/010091, filed on Mar. 8, 2022.

(30) Foreign Application Priority Data
Jun. 10, 2021 (JP) 2021-097391

(51) Int. Cl. C09D 5/16; C07C 19/10; C07C 43/215; C07C 63/34; C07D 207/14; C07D 401/06; C09D 15/00

(52) U.S. Cl. CPC C09D 5/1625; C07C 19/10; C07C 43/215; C07C 63/34; C07D 207/14; C07D 401/06; C09D 15/00

(57) ABSTRACT

Provided is a wood preservation treatment liquid including: an organic solvent containing 1-chloro-2,3,3,3-tetrafluoropropene; and a wood preservative.

### CROSS-REFERENCE TO RELATED APPLICATION

This application is a Continuation of International Application No. PCT/JP2022/010091, filed Mar. 8, 2022, which claims priority to Japanese Patent Application No. 2021-097391 filed Jun. 10, 2021. Each of the above applications is hereby expressly incorporated by reference, in its entirety, into the present application.

### TECHNICAL FIELD

The present disclosure relates to a wood preservation treatment liquid and a method of producing a wood provided with a wood preservative.

### BACKGROUND ART

Woods are materials used in various fields. However, the woods have drawbacks in that the woods are rotted and degraded due to corrosion caused by fungi or damage caused by insects, whereby the strength of the woods is decreased. Thus, various wood preservation treatment liquids for preventing degradation caused by fungi or insects and methods of producing woods provided with wood preservatives using the wood preservation treatment liquids have been conventionally developed.

Japanese Patent Application Laid-Open (JP-A) No. 2021-66174 discloses a chemical treatment agent composition that includes an active constituent that modifies a wood material, and an organic solvent in which the active constituent is dissolved, and which includes a hydrofluoroolefin.

### SUMMARY OF INVENTION

### Technical Problem

One of the performances demanded for a wood preservation treatment liquid is permeation performance into a wood. In a case in which the permeation performance of a wood preservation treatment liquid into a wood is high, the amount of wood preservative introduced into a wood can be increased, and the degradation of a wood can be further suppressed.

The present inventors examined permeation performance into a wood using a hydrofluoroolefin (for example, 1-chloro-3,3,3-trifluoro-1-propene) specifically disclosed in JP-A No. 2021-66174, and thus found that further improvement was necessary.

An object of the present disclosure is to provide a wood preservation treatment liquid excellent in permeation performance into a wood in view of such actual circumstances.

An additional object of the present disclosure is to provide a method of producing a wood provided with a wood preservative.

### Solution to Problem

The present inventors found that the above-described problems can be solved by the following configurations.

(1) A wood preservation treatment liquid including: an organic solvent containing 1-chloro-2,3,3,3-tetrafluoropropene; and a wood preservative.

(2) The wood preservation treatment liquid according to (1), wherein a content of the 1-chloro-2,3,3,3-tetrafluoropropene is 50% by mass or more with respect to a total mass of the organic solvent.

(3) The wood preservation treatment liquid according to (1) or (2), wherein a content of the organic solvent is from 70 to 99.998% by mass with respect to a total mass of the wood preservation treatment liquid.

(4) The wood preservation treatment liquid according to any of (1) to (3), wherein the 1-chloro-2,3,3,3-tetrafluoropropene includes a Z form of the 1-chloro-2,3,3,3-tetrafluoropropene and an E form of the 1-chloro-2,3,3,3-tetrafluoropropene, and a content of the Z form of the 1-chloro-2,3,3,3-tetrafluoropropene is 50% by mass or more with respect to a total amount of the Z form of the 1-chloro-2,3,3,3-tetrafluoropropene and the E form of 1-chloro-2,3,3,3-tetrafluoropropene.

(5) The wood preservation treatment liquid according to any of (1) to (4), wherein the wood preservative is at least one selected from an ammonium-based wood preservative, a triazole-based wood preservative, a neonicotinoid-based wood preservative, a phenylpyrazole-based wood preservative, a phenylpyrrole-based wood preservative, a benzoylphenyl urea-based wood preservative, an anthranilic diamide-based wood preservative, a strobilurin-based wood preservative, a pyrethroid-based wood preservative, a copper or zinc salt of a fatty acid, or a copper or zinc salt of a fatty acid having an aromatic ring.

(6) The wood preservation treatment liquid according to any of (1) to (4), wherein the wood preservative is at least one selected from a triazole-based wood preservative, a neonicotinoid-based wood preservative, a pyrethroid-based wood preservative, a copper or zinc salt of a fatty acid, or a copper or zinc salt of a fatty acid having an aromatic ring.

(7) The wood preservation treatment liquid according to any of (1) to (4), wherein the wood preservative is at least one selected from (2RS,3RS;2RS,3SR)-2-(4-chlorophenyl)-3-cyclopropyl-1-(1H-1,2,4-triazol-1-yl)butan-2-ol, 1-[(6-chloro-3-pyridinyl)methyl]-4,5-dihydro-N-nitro-1H-imidazol-2-amine, 2-(4-ethoxyphenyl)-2-methylpropyl-3-phenoxybenzylether, zinc naphthenate, or copper naphthenate.

(8) The wood preservation treatment liquid according to any one of (1) to (7), wherein a content of the wood preservative is from 0.002 to 30% by mass with respect to the total mass of the wood preservation treatment liquid.

(9) The wood preservation treatment liquid according to any of (1) to (8), wherein the wood preservation treatment liquid does not substantially include water, or a content of water in the wood preservation treatment liquid is 10% by mass or less with respect to the total mass of the wood preservation treatment liquid.

(10) The wood preservation treatment liquid according to any of (1) to (9), wherein a ratio between a mass of treated wood and a mass of pretreatment wood is 3.0 or more in both cases of 0.05 MPaG and 0.10 MPaG in a pressure-resistant container when the ratio is measured based on the following permeation performance test:

(Permeation Performance Test)

a cedar wood piece having a thickness of 15 mm, a width of 15 mm, and a length of 50 mm is put in a glass tube, a wood preservation treatment liquid in an amount allowing a whole of the wood piece to be soaked in the wood preservation treatment liquid is injected into the glass tube, the glass tube is housed in the pressure-resistant container, the pressure-resistant container is sealed, the pressure-resistant container is pressurized using compressed air to achieve 0.05 MPaG or 0.10 MPaG in the pressure-resistant container, and is left to stand for 30 minutes under an environment of 25° C., the wood piece is taken out after the pressure-resistant container has been left to stand, and the ratio of the mass of treated wood, which is a mass of the wood piece just after the wood piece has been taken out, to the mass of pretreatment wood, which is a mass of the wood piece prior to the permeation performance test, is calculated in each of the cases of 0.05 MPaG and 0.10 MPaG in the pressure-resistant container.

(11) A method of producing a wood provided with a wood preservative, including bringing the wood preservation treatment liquid according to any of (1) to (10) into contact with a wood, and volatilizing an organic solvent to produce a wood provided with a wood preservative.

### Advantageous Effects of Invention

In accordance with one embodiment of the present disclosure, a wood preservation treatment liquid excellent in permeation performance into a wood can be provided.

In accordance with one embodiment of the present disclosure, a method of producing a wood provided with a wood preservative can be provided.

### DESCRIPTION OF EMBODIMENTS

One embodiment of the present disclosure is described in detail below.

In the present specification, a numerical range expressed by “x to y” means a range including the values of x and y as the lower and upper limit values, respectively.

In the present specification, the abbreviated name of a compound is indicated in parenthesis following the name of the compound, and the abbreviated name is used instead of the name of the compound, if necessary. (E) and (Z), included in the name of a compound having a geometrical isomer, or the abbreviated name thereof, indicate an E form (trans form) and a Z form (cis form), respectively. In a case in which neither E form nor Z form is specified in the name or abbreviated name of the compound, the name or abbreviated name means a generic name including the E form, the Z form, and the mixture of the E and Z bodies.

Examples of the key points of the wood preservation treatment liquid of the present disclosure include use of 1-chloro-2,3,3,3-tetrafluoropropene (CF₃—CF═CHCl, HCFO-1224yd; hereinafter also simply referred to as “1224yd”). According to an examination performed by the present inventors, it was revealed that the permeation performance of 1224yd into a wood is high, and it was found that the permeation performance of the wood preservation treatment liquid of the present disclosure into a wood is high.

The wood preservation treatment liquid of the present disclosure includes: an organic solvent containing 1224yd; and a wood preservative.

Each constituent is described in detail below.

(Organic Solvent)

The organic solvent contains 1224yd

Such 1224yd may be the Z form (cis form) of 1224yd or the E form (trans form) of 1224yd. The wood preservation treatment liquid of the present disclosure may include only the Z form of 1224yd, may include only the E form of 1224yd, or may include both the Z and E forms of 1224yd.

Especially, the organic solvent preferably includes at least the Z form of 1224yd in view of higher volatility.

The content of the Z form of 1224yd is preferably 50% by mass or more and more preferably 70% by mass or more with respect to the total amount of the Z form of 1224yd and the E form of 1224y in view of maintenance of the excellent volatility of the Z form. Examples of the upper limit thereof include 100% by mass or less.

The content of 1224yd in the organic solvent is preferably 50% by mass or more, more preferably 70% by mass or more, and still more preferably 90% by mass or more with respect to the total mass of the organic solvent. Examples of the upper limit of the content of 1224yd in the organic solvent include 100% by mass or less.

As long as the organic solvent includes 1224yd, the organic solvent may or need not include another solvent.

A solvent in which a wood preservative can be dispersed or dissolved is preferred as the organic solvent.

In a case in which the organic solvent includes an additional organic solvent other than 1224yd, the content of the additional organic solvent is preferably less than 50% by mass, more preferably 30% by mass or less, and still more preferably 10% by mass or less with respect to the total mass of the organic solvent. Examples of the lower limit of the content of the additional organic solvent other than 1224yd in the organic solvent include 0% by mass.

The additional organic solvent may be used singly, or in combination of two or more kinds thereof.

Examples of the additional organic solvents include hydrocarbons, alcohols, ketones, ethers (excluding hydrofluoroethers), esters, nitrogen-containing organic solvents, and halogenated hydrocarbons (excluding 1224yd).

Such a hydrocarbon is preferably chain or cyclic, saturated or unsaturated hydrocarbon having 5 to 8 carbon atoms, and specific examples thereof include n-pentane, 2-methylbutane, n-hexane, 2-methylpentane, 2,2-dimethylbutane, 2,3-dimethylbutane, n-hebutane, 2-methylhexane, 3-methylhexane, 2,4-dimethylpentane, n-octane, 2-methylheptane, 3-methylheptane, 4-methylheptane, 2,2-dimethylhexane, 2,5-dimethylhexane, 3,3-dimethylhexane, 2-methyl-3-ethylpentane, 3-methyl-3-ethylpentane, 2,3,3-trimethylpentane, 2,3,4-trimethylpentane, 2,2,3-trimethylpentane, 2-methylheptane, 2,2,4-trimethylpentane, cyclopentane, methylcyclopentane, cyclohexane, methylcyclohexane, ethylcyclohexane, and cyclohexene. Especially, n-pentane, cyclopentane, n-hexane, cyclohexane, and n-hebutane are more preferred.

Such a hydrocarbon may be a hydrocarbon having 9 or more carbon atoms, such as mineral oil, kerosene, or petroleum benzine. Such a hydrocarbon may be an aromatic hydrocarbon such as toluene or xylene.

Such an alcohol is preferably a chain or cyclic, saturated or unsaturated alcohol having 1 to 6 carbon atoms, and specific examples thereof include methanol, ethanol, n-propyl alcohol, isopropyl alcohol, n-butyl alcohol, sec-butyl alcohol, isobutyl alcohol, tert-butyl alcohol, 1-pentanol, 2-pentanol, 1-ethyl-1-propanol, 2-methyl-1-butanol, 3-methyl-1-butanol, 3-methyl-2-butanol, neopentyl alcohol, 1-hexanol, 2-methyl-1-pentanol, 4-methyl-2-pentanol, 2-ethyl-1-butanol, allyl alcohol, propargyl alcohol, benzyl alcohol, and cyclohexanol. Especially, methanol, ethanol, and isopropyl alcohol are more preferred.

Such a ketone is preferably a chain or cyclic, saturated or unsaturated ketone having 3 to 6 carbon atoms, and specific examples thereof include acetone, methyl ethyl ketone, 2-pentanone, 3-pentanone, 2-hexanone, methyl isobutyl ketone, and cyclohexanone. Especially, acetone and methyl ethyl ketone are more preferred.

Such an ether is preferably a chain or cyclic, saturated or unsaturated ether having 2 to 6 carbon atoms, and specific examples thereof include diethyl ether, dipropyl ether, diisopropyl ether, dimethoxymethane, ethyl vinyl ether, butyl vinyl ether, dioxane, furan, methylfuran, and tetrahydrofuran. Especially, diethyl ether, diisopropyl ether, dimethoxymethane, dioxane, and tetrahydrofuran are more preferred.

Such an ester is preferably a chain or cyclic, saturated or unsaturated ester having 2 to 8 carbon atoms, and specific examples thereof include methyl formate, ethyl formate, propyl formate, butyl formate, isobutyl formate, pentyl formate, methyl acetate, ethyl acetate, propyl acetate, isopropyl acetate, butyl acetate, isobutyl acetate, sec-butyl acetate, pentyl acetate, methoxybutyl acetate, sec-hexyl acetate, 2-ethylbutyl acetate, cyclohexyl acetate, benzyl acetate, methyl propionate, ethyl propanoate, butyl propionate, methyl butyrate, ethyl butylate, butyl butyrate, isobutyl isobutyrate, ethyl 2-hydroxy-2-methylpropionate, methyl benzoate, ethyl benzoate, γ-butyrolactone, diethyl oxalate, diethyl malonate, dimethyl maleate, diethyl maleate, and dimethyl tartrate. Especially, methyl acetate and ethyl acetate are more preferred.

Specific examples of the nitrogen-containing organic solvents include N-methyl-2-pyrrolidone, N-methylpyrrole, dimethylformamide, dimethyl sulfoxide, pyrrole, N-methylpyrrole, N-methylmorpholine, pyridine, and N-methylpyridine.

Examples of the halogenated hydrocarbons (excluding 1224yd) include methylene chloride, trichloroethylene, tetrachloroethylene, 1,2-dichloroethylene (either or both of cis form and trans form), 1-chloro-2,3,3-trifluoro-1-propene (either or both of E form and Z form), 1-chloro-3,3,3-trifluoro-1-propene (either or both of E form and Z form), 1,1,1,4,4,4-hexafluoro-2-butene (either or both of cis form and trans form), 1,1,2,2-tetrafluoroethyl-2,2,2-trifluoroethoxyethane, either or both of 1,1,1,2,2,3,3,4,4-nonafluoro-methoxybutane and 2-trifluoromethyl-1,1,1,2,3,3-nonafluoro-methoxypropane, 1,1,1,2,2,3,3,4,4-nonafluoro-ethoxybutane, 2-trifluoromethyl-1,1,1,2,3,3-nonafluoro-ethoxypropane, 1,1,1-2,3,4,4,5,5,5-decafluoro-3-methoxy-2-(trifluoromethyl)pentane, and the isomers of perfluoroheptene ether.

The content of the organic solvent is preferably from 70 to 99.998% by mass, and more preferably from 80 to 99.99% by mass with respect to the total mass of the wood preservation treatment liquid.

(Wood Preservative)

A wood preservative that has been conventionally commonly used can be used as the wood preservative. The wood preservative may be a wood preservative that is used as a germicide or an insecticide that removes a fungus that corrodes a wood and an insect such as a termite that eats a wood, or as an antiseptic, a repellent for ants, or a fungicide.

The wood preservative is mixed with the organic solvent containing 1224yd, the mixture is injected into a wood, and the organic solvent is then volatilized to allow the wood preservative to remain in the wood.

Such 1224yd has a halogen atom, particularly a chlorine atom, in the molecule thereof, and thus exhibits excellent solubility in the wood preservative described above. It is considered that 1224yd further has an unsaturated bond (carbon-carbon double bond), and therefore has polarity in the molecule thereof.

Therefore, among such wood preservatives, a wood preservative such as an ammonium-based wood preservative, a triazole-based wood preservative, a neonicotinoid-based wood preservative, a phenylpyrazole-based wood preservative, a phenylpyrrole-based wood preservative, a benzoylphenyl urea-based wood preservative, an anthranilic diamide-based wood preservative, a strobilurin-based wood preservative, a pyrethroid-based wood preservative, or a salt of a fatty acid or a fatty acid having an aromatic ring and copper or zinc is considered to have particularly excellent solubility in 1224yd.

Specific examples of the ammonium-based wood preservative include didecyldimethylammonium chloride, N,N-didecyl-N-methyl-polyoxyethyl-ammonium propionate, and N-alkylbenzyldimethylammonium chloride.

Specific examples of the triazole-based wood preservative include (2RS,3RS;2RS,3 SR)-2-(4-chlorophenyl)-3-cyclopropyl-1-(1H-1,2,4-triazol-1-yl)butan-2-ol, (RS)-1-p-chlorophenyl-4,4-dimethyl-3-(1H-1,2,4-triazol-1-ylmethyl)pentan-3-ol, and 1-[[2-(2,4-dichlorophenyl)-4-propyl-1,3-dioxolan-2-yl]methyl]-1H-1,2,4-triazole.

Specific examples of the neonicotinoid-based wood preservative include (E)-N1-[(6-chloro-3-pyridyl)methyl]-N2-cyano-N1-methylacetamidine, 1-[(6-chloro-3-pyridinyl)methyl]-4,5-dihydro-N-nitro-1H-imidazol-2-amine, (E)-1-(2-chloro-1,3-thiazol-5-ylmethyl)-3-methyl-2-nitroguanidine, and (EZ)-3-(2-chloro-1,3-thiazol-5-ylmethyl)-5-methyl-1,3,5-oxadiazinan-4-ylidene(nitro)amine.

Specific examples of the phenylpyrazole-based wood preservative include 5-amino-1-[2,6-dichloro-4-(trifluoromethyl)phenyl]-4-[(trifluoromethyl)sulfonyl]-1H-pyrazole-3-carbonitrile.

Specific examples of the phenylpyrrole-based wood preservative include 4-(2,2-difluoro-1,3-benzodioxol-4-yl)pyrrole-3-carbonitrile.

Specific examples of the benzoylphenyl urea-based wood preservative include 1-[3,5-dichloro-4-(3-chloro-5-trifluoromethyl-2-pyridyloxy)phenyl]-3-(2,6-difluorobenzoyl)urea and (RS)-1-[3-chloro-4-(1,1,2-trifluoro-2-trifluoromethoxyethoxy)phenyl]-3-(2,6-difluorobenzoyl)urea.

Specific examples of the anthranilic diamide-based wood preservative include 3-bromo-N-[4-chloro-2-methyl-6-(methyl carbamoyl)phenyl]-1-(3-chloropyridin-2-yl)-1H-pyrazole-5-carboxamide.

Specific examples of the strobilurin-based wood preservative include methyl={2-[1-(4-chlorophenyl)pyrazol-3-yloxymethyl]phenyl}(methoxy)carbamate.

Specific examples of the pyrethroid-based wood preservative include (±)-α-cyano-3-phenoxybenzyl(+)-cis,trans-chrysanthemate, 2-(4-ethoxyphenyl)-2-methylpropyl-3-phenoxybenzylether, 4-ethoxyphenyl [3-(4-fluoro-3-phenoxyphenyl)propyl] dimethylsilane, 2-methylbiphenyl-3-ylmethyl(Z)-(1RS,3RS)-3-(2-chloro-3,3,3-trifluoroprop-1-enyl)-2,2-dimethylcyclopropanecarboxylate, (Z)—(S)-2-methyl-4-oxo-3-(penta-2,4-dienyl)cyclopent-2-enyl=(1R)-trans-2,2-dimethyl-3-(2-methylprop-1-enyl)cyclopropanecarboxylate, (Z)—(S)-2-methyl-4-oxo-3-(penta-2,4-dienyl)cyclopent-2-enyl=(+)-trans-chrysanthemate, and 3-phenoxybenzyl=2-(2,2-dichlorovinyl)-3,3-dimethyl-1-cyclopropanecarboxylate.

Specific examples of the salt of a fatty acid or a fatty acid having an aromatic ring and copper or zinc include zinc naphthenate, copper naphthenate, tertiary zinc carboxylate, and zinc versatate.

Examples of such additional agents include zinc oxide, cupric oxide, cupric oxide, cupric oxide, cupric hydroxide, 4-bromo-2-(4-chlorophenyl)-1-ethoxymethyl-5-(trifluoromethyl)pyrrole-3-carbonitrile, (EZ)-2′-[2-(4-cyanophenyl)-1-(α,α,α-trifluoro-m-tolyl)ethylidene]-4-(trifluoromethoxy)carbanilohydrazide, (RS)-1-methyl-2-nitro-3-(tetrahydro-3-furylmethyl)guanidine, 3-iodine-2-propynylbutylcarbamate, 2-phenylphenol, and creosote oil containing coal tar as a main constituent.

Among the wood preservatives described above, for example, (2RS,3RS;2RS,3SR)-2-(4-chlorophenyl)-3-cyclopropyl-1-(1H-1,2,4-triazol-1-yl)butan-2-ol (for example, cyproconazole commercially available from FTS Inc.) which is a triazole-based wood preservative, 1-[(6-chloro-3-pyridinyl)methyl]-4,5-dihydro-N-nitro-1H-imidazol-2-amine (for example, imidacloprid commercially available from LANXESS) which is a neonicotinoid-based wood preservative, 2-(4-ethoxyphenyl)-2-methylpropyl-3-phenoxybenzylether (for example, etofenbrox commercially available from Mitsui Chemicals Agro, Inc.) which is a pyrethroid-based wood preservative, zinc naphthenate (for example, a product commercially available from Tokyo Chemical Industry Co., Ltd.), and copper naphthenate (for example, a product commercially available from Show Kagaku K.K.) are preferred from the view point of performance as the wood preservation agents, and dispersibility and solubility with 1224yd.

The wood preservative may be used singly, or in combination of two or more kinds thereof.

The content of the wood preservative is preferably from 0.002 to 30% by mass, and more preferably from 0.01 to 20% by mass with respect to the total mass of the wood preservation treatment liquid.

The wood preservation treatment liquid of the present disclosure may include an additive such as a stabilizer or a dispersant, as well as the organic solvent and wood preservative described above.

The content of the additive is preferably 1% by mass or less with respect to the total mass of the wood preservation treatment liquid.

The wood preservative may include another constituent for the purpose of improving adjustment of viscosity or solubility in an organic solvent in view of handleability.

Specifically, the wood preservative may include a surfactant or water. Examples of the surfactant include nonionic surfactants, anionic surfactants, and cationic surfactants.

The content of the water is preferably 10% by mass or less, more preferably 5% by mass or less, and still more preferably 3% by mass or less with respect to the total mass of the wood preservation treatment liquid, and it is particularly preferable that the wood preservative does not substantially include water. The content of the water is set in the range described above, whereby an increase in the percentage of the content of water in a wood can be suppressed to suppress a variation in the dimension of the wood in a case in which the wood preservation treatment liquid is brought into contact with the wood. In regard to the content of the water, “does not substantially include” refers to not more than a detection limit in a case in which the content is measured by known means based on common general technical knowledge in the art.

<Permeation Performance of Wood Preservation Treatment Liquid>

The permeation performance of the wood preservation treatment liquid of the present disclosure into a wood will now be described.

A ratio between the mass of a treated wood and the mass of a pretreatment wood is preferably 3.0 or more in a case in which the permeation performance of the wood preservation treatment liquid of the present disclosure into a wood is measured based on a permeation performance test described below. In the permeation performance test described below, the ratio is preferably 3.0 or more in either case of 0.05 MPaG or 0.10 MPaG in a pressure-resistant container, and more preferably 3.0 or more in both the cases of 0.05 MPaG and 0.10 MPaG in the pressure-resistant container. Satisfaction of the ratio described above enables production of a wood provided with a wood preservative, of which the interior is sufficiently infiltrated by the wood preservative.

(Permeation Performance Test)

A cedar wood piece having a thickness of 15 mm, a width of 15 mm, and a length of 50 mm is put in a glass tube, the wood preservation treatment liquid in an amount allowing the whole of the wood piece to be soaked in the wood preservation treatment liquid is injected into the glass tube, the glass tube is housed in the pressure-resistant container, the pressure-resistant container is sealed, the pressure-resistant container is pressurized using compressed air to achieve 0.05 MPaG or 0.10 MPaG in the pressure-resistant container, and is left to stand for 30 minutes under an environment of 25° C. Then, the wood piece is taken out after the pressure-resistant container has been left to stand, and the ratio of the mass of treated wood, which is the mass of the wood just after the wood piece has been taken out, to the mass of pretreatment wood, which is the mass of the wood piece prior to the permeation performance test, is calculated in each of the cases of 0.05 MPaG and 0.10 MPaG in the pressure-resistant container.

<Method of Producing Wood Provided with Wood Preservative>

A method of producing a wood provided with the wood preservative of the present disclosure will now be described.

The method of producing a wood provided with the wood preservative of the present disclosure is a production method including bringing the described-above wood preservation treatment liquid according to the present disclosure into contact with a wood, and volatilizing an organic solvent to produce a wood provided with the wood preservative. By bringing the wood preservation treatment liquid of the present disclosure into contact with the wood, the wood preservation treatment liquid of the present disclosure permeates the interior of the wood.

Examples of a method of bringing the wood preservation treatment liquid of the present disclosure into contact with a wood include a method of brushing a wood surface with the wood preservation treatment liquid, a method of spraying the wood preservation treatment liquid on a wood surface, a method of dipping a wood in the wood preservation treatment liquid, and a method of applying pressure in a state in which a wood is dipped in the wood preservation treatment liquid. Especially, the method of applying pressure in a state in which a wood is dipped in the wood preservation treatment liquid is preferred.

The wood preservative in the wood preservation treatment liquid is preferably dissolved in the above-described organic solvent containing 1224yd. Therefore, the treatment described above allows the wood preservative, together with the organic solvent containing 1224yd, to adhere to the surface of the wood, and to further infiltrate the interior of the wood from the vicinity of the surface of the wood. Then, the organic solvent is volatilized and removed from the interior of the wood by heating the wood, leaving the wood to stand at ordinary temperature, or depressurizing the wood.

In a case in which such an organic solvent is volatilized and removed from the interior of the wood, the organic solvent may be dissolved in water in the wood, whereby removal performance deteriorates. In this regard, the Z form of 1224yd with low solubility in water is preferred.

For example, a method described in Japanese Patent No. 4149422 can be utilized as the treatment method of applying pressure in a state in which a wood is dipped in the wood preservation treatment liquid, to allow the wood preservation treatment liquid to permeate the wood, among the methods described above. The 1224yd used in the present disclosure is easily collected by evaporation and condensation because the 1224yd is a volatile liquid of which the boiling point is from 10 to 30° C. at ordinary pressure even in the case of using the 1224yd in such a treatment apparatus as described in Japanese Patent No. 4149422.

In such a method of preservation treatment of a wood as described in Japanese Patent No. 4149422, the preservation treatment is commonly performed in a closed system, and therefore, a wood preservation treatment liquid may be moved between a container for treatment of a wood and a preservation container such as a tank in which the wood preservation treatment liquid is preserved. In such a case, 1224yd has a vapor pressure that is not less than atmospheric pressure at ordinary temperature, and therefore, the wood preservation treatment liquid of the present disclosure facilitates the generation of a differential pressure between the containers by controlling pressure and temperature. For example, a pressure at the time of pressurization in the case of moving the wood preservation treatment liquid by a differential pressure between the containers due to the pressurization is less than that in the case of using an organic solvent having a pressure that is less than atmospheric pressure at ordinary temperature. Alternatively, in the case of moving the wood preservation treatment liquid by warming or cooling either or both of the treatment container and the preservation container, a differential pressure between the containers is greater than that in the case of using an organic solvent having a pressure that is less than atmospheric pressure at ordinary temperature, and the wood preservation treatment liquid can be moved only by temperature control.

The pressure control and the temperature control may be combined.

Thus, movement of the wood preservation treatment liquid due to the differential pressure of 1224yd is facilitated, and therefore, the energy of transportation by a transportation pump can be reduced.

Further, in view of collection of the organic solvent released from the wood impregnated with the wood preservation treatment liquid, 1224yd having a boiling point of 15° C., used in the present disclosure, is easily volatilized from the wood, and therefore, the amount of 1224yd released into the environment can be reduced by devising a collection apparatus. In addition, 1224yd itself has low environmental impact, and therefore, even in a case in which 1224yd is released into the environment, an environmental load can be reduced in comparison with a case in which another organic solvent is released.

### EXAMPLES

One embodiment of the present disclosure is described in more detail below with reference to Examples; however, the present disclosure is not limited thereto. Example 1 corresponds to an example, and Example 2 corresponds to a comparative example.

According to a method described in WO 2017/110851, 1224yd was produced as an isomer mixture of HCFO-1224yd (E) and HCFO-1224yd (Z). The isomer mixture was purified by a method described in WO 2017/146190, to produce HCFO-1224yd (E) and HCFO-1224yd (Z). HCFO-1224yd used in Examples was mixed so that a mass ratio represented by HCFO-1224yd (Z)/HCFO-1224yd (E) was 99/1. Thus, purified HCFO-1224yd having a purity of 99.5% was obtained.

In the following test, the purified 1224yd is referred to as “1224yd”.

The Z form of 1233zd was synthesized by a method described in JP-A No. 2013-87066. In other words, crude 1233zd (Z) including 3-chloro-1,1,1,3-tetrafluoropropane (HCFC-244fa), 2-chloro-1,1,1,3,3-pentafluoropropane (HCFC-235da), and 1233zd (Z) was mixed with triethylamine, and the mixture was allowed to react at 150° C. for 3 hours, to obtain a reaction product including 1233zd (Z). The reaction product was further purified to obtain purified 1233zd (Z) having a purity of 99.5%. In the following test, the purified 1233zd (Z) was referred to as “1233zd (Z)”.

A zinc naphthenate/mineral spirit solution (Zn: 8%) which was a reagent manufactured by FUJIFILM Wako Pure Chemical Corporation was used as a wood preservative.

### Examples 1 to 2

The wood preservative described above was dissolved in each solvent as set forth in Table 1, to prepare a wood preservation treatment liquid.

<Permeation Performance Test>

By the following method, a wood was dip-treated using the prepared wood preservation treatment liquid.

A cedar wood piece having a thickness of 15 mm, a width of 15 mm, and a length of 50 mm, cut from a wood sheet having a thickness of 15 mm, was put in a glass tube, the wood preservation treatment liquid of Example 1 or 2 was injected into the glass tube, the glass tube was then housed in a pressure-resistant container, the pressure-resistant container was sealed, the interior of the pressure-resistant container was pressurized to a pressure set forth in Table 1 by compressed air, and the pressure-resistant container was left to stand for 30 minutes.

Then, the wood preservation treatment liquid in the pressure-resistant container was transferred and collected into another container, and the pressure of the pressure-resistant container was released to atmospheric pressure to obtain a wood provided with a wood preservative before drying.

A variation between the mass of the wood provided with a wood preservative before drying, obtained by the method described above, just after the wood was taken out of the pressure-resistant container (mass of treated wood) and the mass of the wood in an early period before permeation treatment (mass of pretreatment wood) was regarded as the permeation performance of the wood preservation treatment liquid, and the permeation performance was evaluated based on the following criteria. A ratio between the mass of the treated wood and the mass of the pretreatment wood is described below. The higher ratio means that a larger amount of wood preservation treatment liquid permeates the wood, and the permeation performance of the wood preservation treatment liquid into the wood is higher.

A: The ratio between the mass of the treated wood and the mass of the pretreatment wood is 3.0 or more.

B: The ratio between the mass of the treated wood and the mass of the pretreatment wood is from 2.5 to less than 3.0.

C: The ratio between the mass of the treated wood and the mass of the pretreatment wood is less than 2.5.

TABLE 1

| | | Wood preservative | | | |
|---|---|---|---|---|---|
| | | | Content of wood | | |
| | | | preservative with respect | | |
| | | | to total mass of wood | Applied | Permeation |
| | Organic solvent | | preservation treatment | pressure | performance |
| Table 1 | Kind | Kind | liquid (% by mass) | (MPaG) | test |
| Example 1 | 1224 yd | Zinc naphthenate | 5.00 | 0.05 | A |
| | | | | 0.10 | A |
| Example 2 | 1233 zd (Z) | Zinc naphthenate | 5.00 | 0.05 | C |
| | | | | 0.10 | B |

The results are set forth in Table 1. The wood preservation treatment liquid of the present disclosure was confirmed to be excellent in permeation performance into a wood.

The permeation performance test was conducted in a procedure similar to that in Example 1 using copper naphthenate instead of zinc naphthenate, and the same results as those in Example 1 were obtained.

A wood provided with a wood preservative, of which the interior is sufficiently infiltrated by a wood preservative, can be produced by drying the wood provided with a wood preservative before drying, obtained by <Permeation Performance Test>, to remove the organic solvent.

The entire disclosure of Japanese Patent Application No. 2021-097391 filed on Jun. 10, 2021 is incorporated herein by reference. All documents, patent applications, and technical standards described in this specification are herein incorporated by reference to the same extent as if each individual document, patent application, or technical standard was specifically and individually indicated to be incorporated by reference.

1. A wood preservation treatment liquid, comprising:
an organic solvent containing 1-chloro-2,3,3,3-tetrafluoropropene; and
a wood preservative.

2. The wood preservation treatment liquid according to claim 1, wherein a content of the 1-chloro-2,3,3,3-tetrafluoropropene is 50% by mass or more with respect to a total mass of the organic solvent.

3. The wood preservation treatment liquid according to claim 1, wherein a content of the organic solvent is from 70 to 99.998% by mass with respect to a total mass of the wood preservation treatment liquid.

4. The wood preservation treatment liquid according to claim 1, wherein:
the 1-chloro-2,3,3,3-tetrafluoropropene comprises a Z form of the 1-chloro-2,3,3,3-tetrafluoropropene and an E form of the 1-chloro-2,3,3,3-tetrafluoropropene, and
a content of the Z form of the 1-chloro-2,3,3,3-tetrafluoropropene is 50% by mass or more with respect to a total amount of the Z form of the 1-chloro-2,3,3,3-tetrafluoropropene and the E form of 1-chloro-2,3,3,3-tetrafluoropropene.

5. The wood preservation treatment liquid according to claim 1, wherein the wood preservative is at least one selected from an ammonium-based wood preservative, a triazole-based wood preservative, a neonicotinoid-based wood preservative, a phenylpyrazole-based wood preservative, a phenylpyrrole-based wood preservative, a benzoylphenyl urea-based wood preservative, an anthranilic diamide-based wood preservative, a strobilurin-based wood preservative, a pyrethroid-based wood preservative, a copper or zinc salt of a fatty acid, or a copper or zinc salt of a fatty acid having an aromatic ring.

6. The wood preservation treatment liquid according to claim 1, wherein the wood preservative is at least one selected from a triazole-based wood preservative, a neonicotinoid-based wood preservative, a pyrethroid-based wood preservative, a copper or zinc salt of a fatty acid, or a copper or zinc salt of a fatty acid having an aromatic ring.

7. The wood preservation treatment liquid according to claim 1, wherein the wood preservative is at least one selected from (2RS,3RS,2RS,3SR)-2-(4-chlorophenyl)-3-cyclopropyl-1-(1H-1,2,4-triazol-1-yl)butan-2-ol, 1-[(6-chloro-3-pyridinyl)methyl]-4,5-dihydro-N-nitro-1H-imidazol-2-amine, 2-(4-ethoxyphenyl)-2-methylpropyl-3-phenoxybenzylether, zinc naphthenate, or copper naphthenate.

8. The wood preservation treatment liquid according to claim 1, wherein a content of the wood preservative is from 0.002 to 30% by mass with respect to a total mass of the wood preservation treatment liquid.

9. The wood preservation treatment liquid according to claim 1, wherein the wood preservation treatment liquid does not substantially comprise water, or a content of water in the wood preservation treatment liquid is 10% by mass or less with respect to a total mass of the wood preservation treatment liquid.

10. The wood preservation treatment liquid according to claim 1, wherein a ratio between a mass of treated wood and a mass of pretreatment wood is 3.0 or more in respective cases of 0.05 MPaG and 0.10 MPaG in a pressure-resistant container when the ratio is measured based on the following permeation performance test:
a cedar wood piece having a thickness of 15 mm, a width of 15 mm, and a length of 50 mm is put in a glass tube, a wood preservation treatment liquid in an amount allowing a whole of the wood piece to be soaked in the wood preservation treatment liquid is injected into the glass tube, the glass tube is housed in the pressure-resistant container, the pressure-resistant container is sealed, the pressure-resistant container is pressurized using compressed air to achieve 0.05 MPaG or 0.10 MPaG in the pressure-resistant container, and is left to stand for 30 minutes in an environment of 25° C., the wood piece is taken out after the pressure-resistant container has been left to stand, and the ratio of the mass of treated wood, which is a mass of the wood piece just after the wood piece has been taken out, to the mass of pretreatment wood, which is a mass of the wood piece prior to the permeation performance test, is calculated for each of the cases of 0.05 MPaG and 0.10 MPaG in the pressure-resistant container.

11. A method of producing wood applied with a wood preservative, comprising bringing the wood preservation treatment liquid according to claim 1 into contact with wood, and volatilizing an organic solvent to produce wood applied with a wood preservative.
